(19) United States
(12) Patent Application Publication
(10) Pub. No.: US 2012/0216601 A1
(43) Pub. Date: Aug. 30, 2012

(54) MICROFLUIDIC DEVICES FOR ANALYZING CELL MOTILITY

(75) Inventor: Daniel Irimia, Charlestown, MA (US)

(73) Assignee: THE GENERAL HOSPITAL CORPORATION, Boston, MA (US)

(21) Appl. No.: 13/407,596

(22) Filed: Feb. 28, 2012

Related U.S. Application Data
(60) Provisional application No. 61/447,240, filed on Feb. 28, 2011.

(51) Int. Cl. G01N 33/48; B65G 53/00

(52) U.S. Cl. 73/53.01; 406/197

(57) ABSTRACT

Methods, apparatus, and systems for analyzing the motility of motile cells, such as neutrophils, are described. The microfluidic devices described herein generally include a main channel and one or more side channels and can be used for causing migration of cells in response to a biochemical gradient. The main channel has an inlet and an outlet and can be sized to receive the cells. The side channels that branch from the main channel terminate in a closed end and are sized such that the cell migrates into and through the side channel in response to a concentration gradient established between the main channel and the interior of the side channels in which the biochemical stimulus is trapped after priming and loading.

### CLAIM OF PRIORITY

This application claims the benefit of U.S. Provisional Patent Application Ser. No. 61/447,240, filed on Feb. 28, 2011, the entire contents of which are hereby incorporated by reference.

### FIELD OF THE INVENTION

This specification relates to analysis of biological samples.

### BACKGROUND

Neutrophil functioning is essential for our well-being and protection against many infectious agents from our close environment. Without neutrophils, we could only survive a few days the constant assault of bacteria and fungi in our normal environment. A tremendous selective pressure on neutrophils to function perfectly in a large number of conditions has made neutrophils one of the most efficient and remarkable cells in terms of migration speed and ability to reach distant targets. However, there are several conditions where neutrophil activity could produce more damage than benefits. While neutrophil activation is protective after minor trauma, hyper-active neutrophils after major injuries have systemic deleterious effects and can effectively damage several organs and tissues, even in the absence of infection. Many conditions like chronic inflammatory diseases, immune reactions post-organ transplantation, or severe forms of asthma can be exacerbated by active neutrophils. Other times, neutrophils become unresponsive, simultaneously with down-regulation of the immune system, leading to, or facilitating septic states. Despite tremendous advances in the understanding of signaling molecules and pathways acting inside neutrophils, our understanding of the changes in neutrophils during disease processes is limited, and consequently, or abilities to modulate the activity of neutrophils in health and disease, restricted to very few options.

Thermal injury triggers a fulminant inflammatory cascade that heralds shock, end-organ failure, and ultimately sepsis and death. Evidence indicates that, in addition to such post-burn inflammatory changes, the motility of neutrophils is also affected. Impairments in neutrophil function after burn injury have potential implications for the development of sepsis. Flow cytometry analysis of neutrophils has shown impairments in phagocytosis, bactericidal activity, phago-lysosomal activity, and the oxidative burst within two weeks post-injury. Neutrophils demonstrate impaired adhesion and complement receptor expression after burn injury, and these changes, in turn, correlate with an increased incidence of abscess formation in vitro.

However, prognostic and diagnostic studies of neutrophil motility have been difficult due to the cumbersome assays that have prohibited exploration of the significance of neutrophil motility after burn injury.

### SUMMARY

This specification describes microfluidic devices for analyzing one or more motile cells, e.g., neutrophil motility.

One example of the microfluidic devices described herein include a substrate in which a main channel is manufactured. Multiple side channels are formed in the substrate such that each side channel branches off the main channel. The side channels can be filled with a chemokine solution. After a motile cell, e.g., a neutrophil, is obtained from a blood sample, it is introduced into a side channel in a non-chemokine suspension, the neutrophil migrates through the channel due to a chemotaxis effect, i.e., a movement of a motile cell in response to a chemical stimulus. The motility of the neutrophil, as the neutrophil migrates through the side channel, can be measured. Such a microfluidic device can be used for prognostic and diagnostic studies of neutrophil motility, for example, in burn victims.

Various aspects of the invention are summarized as follows. In general, in a first aspect, the subject matter of the disclosure can be embodied in methods for manipulating cells in a chemo-attractant environment, in which the methods include filling a main channel and a side channel branching off of the main channel with a first fluid that includes at least one chemokine, and introducing a second fluid free of the at least one chemokine and that includes multiple cells into the main channel such that an individual cell of the multiple cells can enter and form a plug in the side channel such that a chemokine concentration gradient is formed in the side channel on opposite sides of the individual cell.

Implementations of the methods can include one or more of the following features and/or features of other aspects. For example, filling can include applying pressure at the main channel inlet that is sufficient to move the first fluid through the main channel and into the side channel. Applying pressure can include applying sufficient pressure to displace air in the side channel through a gas permeable substrate defining a portion of the side channel.

In some implementations, the methods further include filling the side channel with a gas that is soluble in the first fluid prior to filling the side channel with the first fluid.

In some instances, filling the side channel with the first fluid includes flowing the first fluid through the main channel past the side channel inlet, in which case the first fluid fills the side channel by capillary action.

In certain aspects, the subject matter of the present disclosure can be embodied in devices for analyzing migration of cells in a chemo-attractant concentration gradient, in which the devices each include: a main channel having a pair of side walls, a main channel inlet, and a main channel outlet, in which the main channel is sized to receive the cells; and a side channel that branches from the main channel, the side channel having an inlet formed in a side wall of the main channel and terminating in a closed end, in which the side channel is sized such that the cells can migrate from the main channel into and form a plug in the side channel and then move along the side channel in the present of a chemo-attractant concentration gradient established in the side channel.

Implementations of the devices can include one or more of the following features and/or features of other aspects. For example, the main channel and the side channel can be positioned on a substrate that includes a gas-permeable material.

In some implementations, the main channel has a cross-sectional area sufficient to allow the cells to flow through. In certain cases, the side channel has a cross-sectional area that is smaller than a cross-sectional area of the main channel. In some implementations, the side channel can be sized such that a cell forms a plug within the side channel. In certain implementations, the side channel is rectangular in cross-section, in which sides of the rectangular cross-section are about 3 μm and 6 μm.

In some implementations, the device further includes multiple side channels, each of which branches from the main channel and has a corresponding inlet formed on the side wall of the main channel. The side channels can branch in a direction that is perpendicular to an axis of the main channel between the main inlet and the main outlet.

In some cases, the side channel is a straight channel or in other implementations, the side channel can include a bend between the first end and the second end.

In certain implementations, the side channel can include a first portion that includes the inlet and a first end, and multiple second portions, each including a second end and terminating at a corresponding closed end, wherein each second end is connected to the first end.

In certain aspects, the subject matter of the present disclosure can be embodied in systems for analyzing target cells, in which each system includes a device having a main channel with a pair of sidewalls, a main channel inlet and a main channel outlet, and multiple side channels, each side channel having an inlet connected to a side wall of the main channel, in which each side channel terminates in a corresponding closed end, and cells are introduced through the main channel inlet and migrate through the multiple side channels. The system can also include a detection unit configured to detect a migration of the cells through the device.

In some implementations, the device includes a gas permeable substrate defining at least part of the side channels. In some other implementations, a cross-sectional area of each side channel is less than a cross-sectional area of cells that migrate through the side channel.

In certain implementations, the main channel has a rectangular cross-section and/or each side channel has a rectangular cross-section.

In some implementations, the main channel and the multiple side channels are microfluidic channels and the multiple side channels can be perpendicular to the main channel.

In some instances, a side channel is square in cross-section, and a side of the square cross-section is about 6 μm. In certain implementations, a side channel is sized such that the cell forms a plug in the side channel.

In additional aspects, the subject matter of the present disclosure is embodied in methods for analyzing target cells, in which the methods include: determining a first migration rate of control cells through a side channel having an inlet and terminating in a dead end, in which the control cells migrate through the side channel due to a chemokine concentration gradient, and the first migration rate includes a distance traveled by a control cell in the side channel per unit time; determining a second migration rate of target cells through the side channel, in which the target cells migrate through the side cells due to the chemokine concentration gradient, and the second migration rate includes a distance traveled by a target cell in the side channel per unit time; and analyzing the target cells by comparing the first migration rate and the second migration rate.

In some implementations of the new methods, determining the first migration rate includes filling the side channel with a first fluid that includes chemokines, introducing a second chemokine-free fluid that includes the control cell at the inlet establishing a chemokine concentration gradient in the side channel, in which the control cell migrates towards a region of high chemokine concentration in the side channel, and measuring the distance traveled by the control cell in the side channel per unit time.

In other aspects, the subject matter of the present disclosure is embodied in methods of modulating neutrophil motility within a patient, in which the methods include obtaining a first neutrophil sample from the patient, the first neutrophil sample including a first neutrophil, determining a first motility of the first neutrophil, and administering a medication that affects neutrophil motility to vary the first motility of the first neutrophil.

Implementations of the methods of modulating motile cell, e.g., neutrophil, motility can include one or more of the following features and/or features of other aspects. For example, administering the medication can include administering a quantity of the medication such that a motility of the neutrophil measured after the medication is administered is greater than (or less than) the first motility. In certain cases, determining the first motility of the first neutrophil includes introducing the first neutrophil to a device as described herein.

For example, the device can include a main channel having a main channel inlet and a main channel outlet connected by a side wall, and a side channel having an inlet connected to the side wall, in which each side channel terminates in a corresponding closed end, and the first neutrophil sample including the first neutrophil is introduced through the main channel, where prior to introducing the neutrophil in the device, a chemokine concentration gradient is established in the side channel, and where the first neutrophil migrates through the side channel due to the chemokine concentration gradient. Determining the first motility can further include measuring a motility of the first neutrophil through the side channel in the chemokine concentration gradient.

In some implementations, the first motility is the motility of the first neutrophil through the side channel.

In some instances, the methods further include obtaining a second neutrophil sample from the patient, the second neutrophil sample including a second neutrophil, in which prior to obtaining the second neutrophil sample, the medication that affects neutrophil motility has been administered to the patient; introducing the second neutrophil sample in the device; obtaining a second motility value of the second neutrophil sample in the side channel; and comparing the first and second motility values. The methods can further include varying the medication based on a result of the comparing.

Implementations of the subject matter described here can provide several advantages. For example, the microfluidic devices for measuring cell, e.g., neutrophil, motility are simple to operate and enable the measurement of neutrophil migration and persistence characteristics at single-cell resolution. The microfluidic devices described here enable the direct observation of moving cells. Consequently, the devices can be easily implemented in a clinical setting, and the prognostic potential of neutrophil motility in a chemokine environment can be explored. Because the neutrophils migrate under an effect of a chemokine gradient established within the microfluidic device, the requirement for expensive equipment to effect flow, for example, syringe pumps, can be decreased or eliminated. Further, the training required to educate an operator to use the microfluidic devices described herein can also be decreased.

The devices can utilize small volumes of blood, a practically important feature to avoid iatrogenic anemia by repeated blood draws from critically ill or pediatric patients. The devices can yield data in less than three hours, and their operation requires, in some embodiments, only the sequential injection of two solutions through the same port of the device, without syringe pumps or complicated priming. In addition, the devices can provide temporal information at the single-cell level, and while most of the motile cells, such as neutrophils, migrate at a steady speed along distinct channels of the devices, their migration speed can be measured to a high degree of precision. The mechanical confinement of neutrophils in the small channels in these devices can also facilitate persistent movement and can have practical consequences on the ease of quantification of motility.

Using the new devices, a reference set of values characteristic of motile cells, e.g., neutrophils, from healthy subjects can be easily obtained. Once a range of “normal values” has been established, these could be used as reference. The reference values are specific for combinations of side-channel size, extracellular matrix coating the channels, and chemokines. For example, the reference value for neutrophil migration in 3×6 μm channels coated with fibronectin and filled with 100 nM fMLP is 18±3 μm/minute. Similarly obtained migration speed values from burn patients and other patients can be compared with the reference values. Data collected in this manner can serve as an early indicator for concurrent infections. Complex relationships between impaired neutrophil motility and the clinical evolution of burn patients can be studied.

The devices can detect significant impairment of motile cell, e.g., neutrophil, migration speed as early as 24 hours after patient admission to the hospital. The ability to measure precisely the neutrophil migration speed independently of all the other parameters of chemotaxis can establish a strong correlation between the depression of migration speed at 72 hours after burn correlated and the total body surface area (TBSA) of burn injury. Overall, the microfluidic devices described herein can contribute to a better understanding of the context of neutrophil pathology associated with the burn injury and its contribution to the septic and other complications in burn patients.

In addition to their use for measuring motile cell, e.g., neutrophil, migration speed, the devices can also be used to measure directional persistence that represents a duration and a distance that a cell moves in one direction before stopping or turning. For example, cells that are motile, but have no directionality, may enter the side channels, but may move back and forth. Alternatively, or in addition, cells following chemo-attractant signals may enter the channels and move up the gradient without stopping or turning. Such directional persistence of cells can be measured and any bias can be designated with numbers that represent the persistence.

As used herein, “motility” means the ability of a motile cell to move, e.g., at a specific migration rate, at least under certain conditions. Motile cells include neutrophils and other immune cells such as granulocyte, monocytes, and lymphocytes, as well as certain cells that can move only under certain specific conditions, such as mast cell, fibroblasts, and endothelial cells (e.g., circulating endothelial cells (“CEC”)) or cells under pathological conditions, such as metastatic cancer cells (e.g., circulating tumor cells (“CTC”)). Sperm and many bacteria are also motile cells. A list of some examples of motile cells that can be monitored using the devices described herein is included in Table 1 below.

As used herein, a “chemokine” means a protein capable of inducing a chemotaxis effect in a cell.

As used herein, “chemotaxis” means a movement of a motile cell in response to a chemical stimulus.

As used herein, “chemo-attractant” means a chemical agent that induces a motile cell to migrate towards the chemical agent.

As used herein, “gas-permeable” means having openings that allow gas to pass through.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are illustrative only and not intended to be limiting.

Other features and advantages of the invention will be apparent from the following detailed description and drawings, and from the claims.

### DESCRIPTION OF DRAWINGS

FIGS. 1A-1F are schematics of examples of a microfluidic device for analyzing neutrophil motility.

FIG. 1G is a photograph of an example of a microfluidic device.

FIG. 2 is a schematic of an example of a first implementation of a microfluidic device having a main channel and multiple side channels.

FIG. 3 is a schematic of an example of a second implementation of a microfluidic device having a main channel and multiple side channels.

FIG. 4 is a schematic of an example of a second implementation of a microfluidic device having a main channel and multiple side channels.

FIG. 5 is a schematic of an example of a third implementation of a microfluidic device having a main channel and multiple side channels.

FIG. 6 is a schematic of an example of a system for determining migration speeds of neutrophils.

FIG. 7 is a flowchart of an example of a process for detecting neutrophil motility using a microfluidic device.

FIG. 8A is a plot of displacement of multiple neutrophils along corresponding channels within the microfluidic device over time.

FIG. 8B is a bar graph of the distribution of average speed of migration for 800 neutrophils from a healthy donor.

FIG. 9 is a flowchart of an example of a process for analyzing migration rates of motile cells.

FIGS. 10A and 10B are a plot of average neutrophil motility in 18 healthy donors and a validation of the repeatability of neutrophil motility, respectively.

FIG. 11 is a plot showing a correlation between neutrophil motility and total burn surface area in burn patients.

FIG. 12 is a plot showing a correlation between neutrophil motility and blood temperature.

FIG. 13 is a flowchart of an example of a process for altering neutrophil motilities in a patient.

FIGS. 14A-B are schematics of examples of a passive micro-valve.

FIGS. 15A-C are schematics of examples of bifurcated side channels.

FIG. 16 is a schematic of an example of a system for switching chemokine gradients.

FIG. 17A is a schematic of an example of a channel for analyzing chemotaxis migration from whole blood.

FIG. 17B is a photograph of an example channel used for analyzing chemotaxis migration from while blood.

FIG. 18 is a photograph of neutrophils migrating from left to right in response to a chemo-attractant.

Like reference symbols in the various drawings indicate like elements.

### DETAILED DESCRIPTION

The microfluidic devices described with reference to the following figures enable the investigation of the details of cell motility, such as neutrophil chemotaxis in burn patients. In particular, the no-flow microfluidic devices can be used to measure the directional migration speed of motile cells, such as neutrophils, in a chemoattractant gradient, with high throughput, and at single cell resolution. The devices were used to measure the migration speed of neutrophils in blood samples from healthy volunteers and to establish a set of reference values for healthy persons regardless of age and sex. Using the new devices, the impairment of cell motility, such as the impairment of neutrophil migration speed after burn injury, can be determined with a high degree of precision.

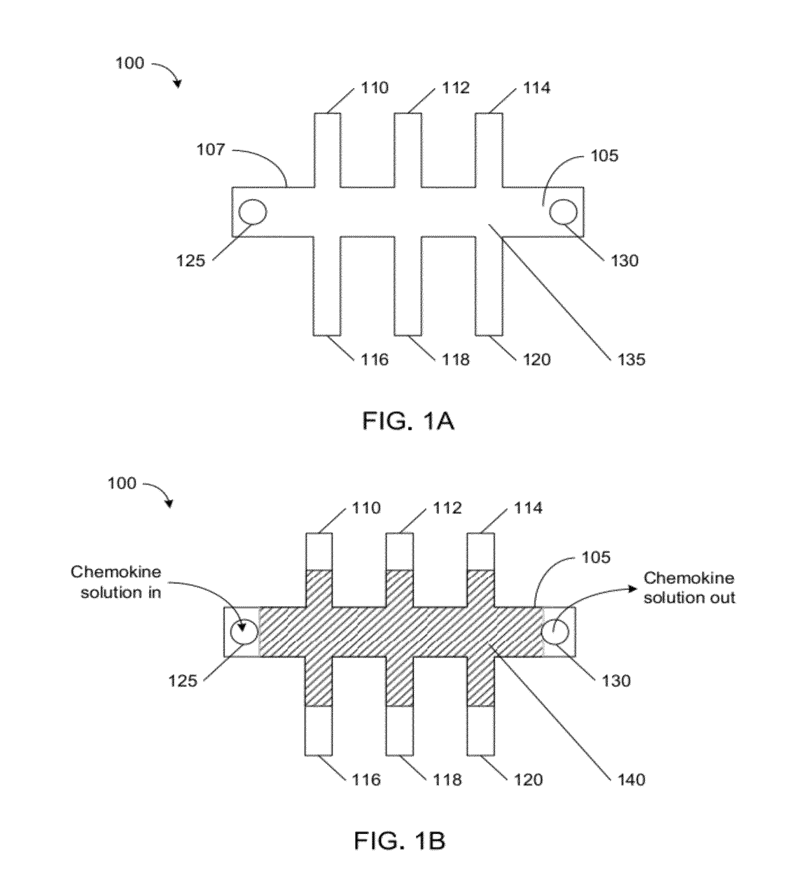

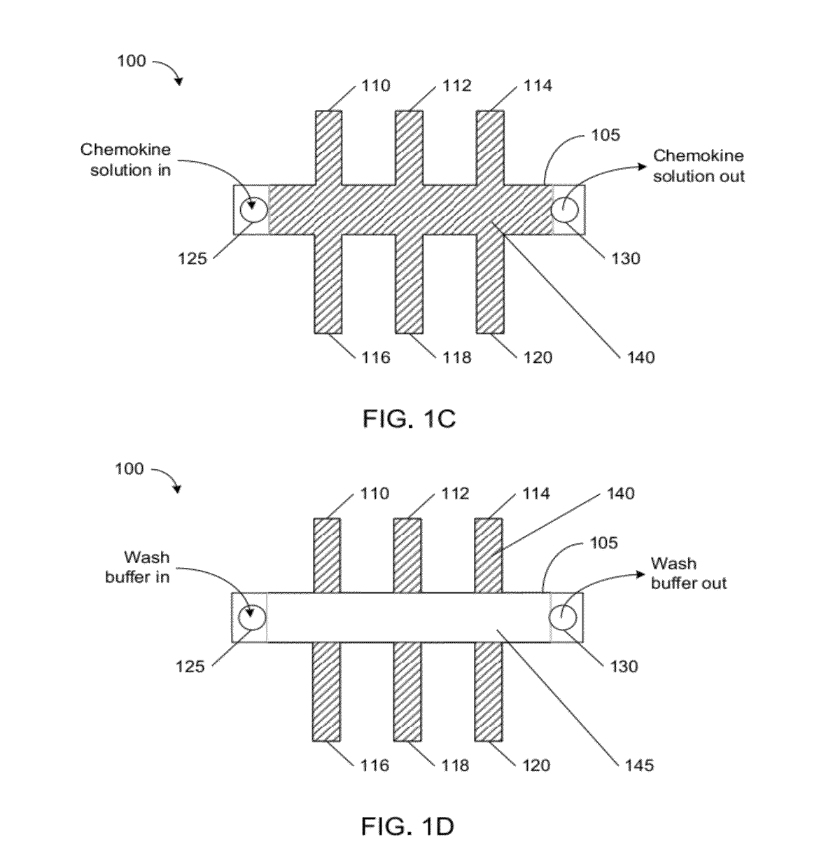

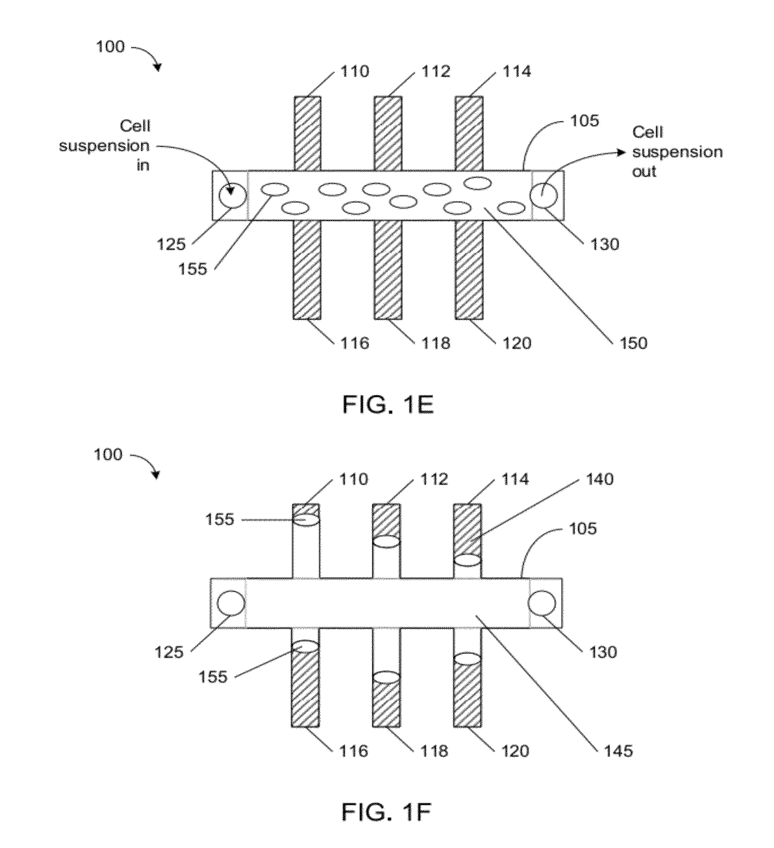

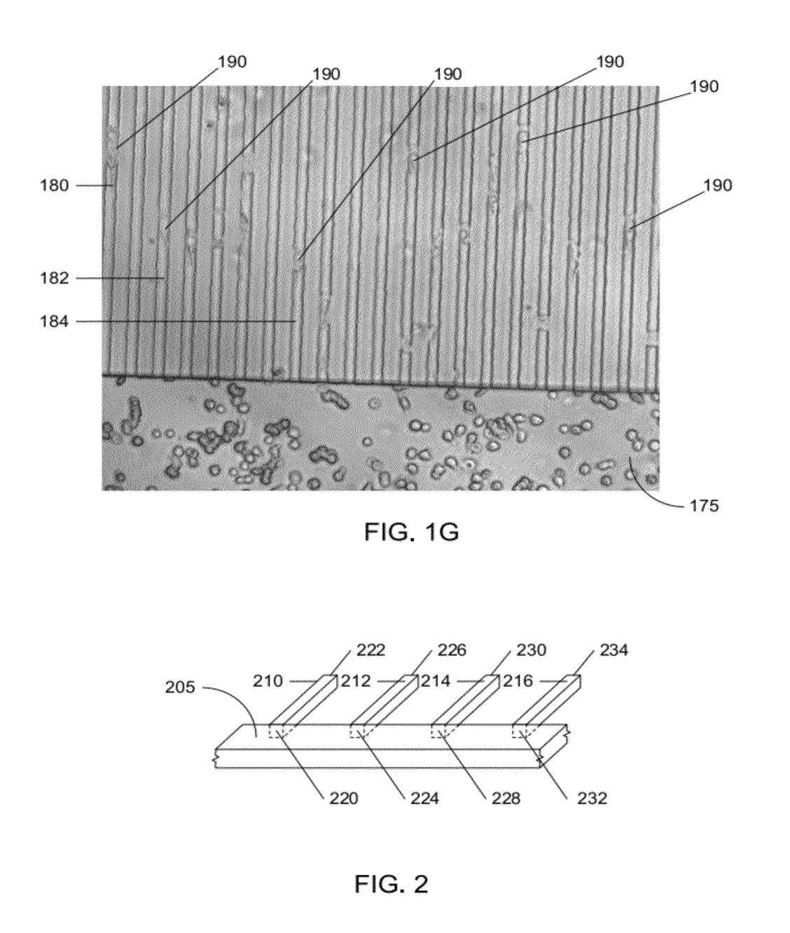

Results obtained using the devices showed that neutrophil motility is depressed as early as 24 hours after the burn injury and is inhibited the most at three to five days after injury. Further, the degree of neutrophil directional migration speed inhibition in burn patients correlates with the magnitude of burn trauma. The migration speed did not correlate with clinical parameters frequently used to monitor these patients, for example, absolute neutrophil numbers, age of circulating neutrophils, or the body temperature. An example of a microfluidic device that was used to obtain these results is described with reference to FIGS. 1A-1G.

FIGS. 1A-1G are schematics of examples of a microfluidic device 100 for analyzing neutrophil motility. The microfluidic device 100 (FIG. 1A) can be used for causing migration of cells, for example, neutrophils, in a chemokine gradient. The device 100 includes a main channel 105 that includes a main channel inlet 125 and a main channel outlet 130 that are connected by a side wall 107. The main channel is sized to receive motile cells, for example, neutrophils suspended in a fluid, as described later.

The device 100 further includes side channels (110, 112, 114, 116, 118, 120). Each side channel, for example, side channel 110, branches from the main channel 105. Each side channel has an inlet formed in the side wall of the main channel and terminates in a closed end. The side channel is sized such that the cells, for example, the neutrophils, migrate through the side channel due to a chemokine concentration gradient established in the side channel. Methods to establish a chemokine concentration gradient in a side channel are described later.

The microfluidic device 100 can be manufactured using the following methods. Two layer of photoresist (SU8, Microchem, Newton, Mass.), the first one 3 μm thin and the second one 50 μm thick, can be patterned on a silicon wafer by sequentially employing two photolithography masks and can be processed using known methods. The wafer with patterned photoresist can be used as a mold to produce PDMS (Polydimethylsiloxane, Fisher Scientific, Fair Lawn, N.J.) parts, which can then be bonded irreversibly to standard glass slides (1×3 inches, Fisher).

Thus, the example microfluidic device 100, described above, includes a first glass substrate and a second PDMS substrate in which the main channel 100 and the multiple side channels (110, 112, 114, 116, 118, 120) can be formed. The inlet 125 and the outlet 130 can be formed either in the first glass substrate or the second PDMS substrate. In other implementations, both substrates can be PDMS substrates or other similar materials.

In general, the substrate in which the main channels and the side channels are formed should be selected to have the following characteristics. The substrate can be gas-permeable so that, when the side channel is filled with a fluid, air in the side channel can be displaced through the substrate as the fluid is pumped in. Alternatively, or in addition, the substrate can be selected so as to facilitate the filling of the side channel with a fluid by capillary action. Furthermore, the substrate can be transparent so as to facilitate image capture of neutrophil motility within the side channels. In some situations, the substrate can be selected such that its surfaces can be coated with agents, for example, proteins, glycoproteins, antibodies, or combinations of them. Such coating can prevent the absorption of soluble factors to the surfaces, and facilitate cell migration, capture of cells in the main channel from cell suspension, and the like.

When formed, the main channel 105 and the side channels (110, 112, 114, 116, 118, 120) are filled with air 135. As shown in FIG. 1B, a chemokine solution 140 can be flowed through the microfluidic device 100. For example, a chemokine solution including the chemokine fMLP [100 nM] and the extracellular matrix protein fibronectin [100 nM] can be flowed into the inlet 125 of the main channel 105, through the main channel 105, and out of the outlet 130, about 15 minutes before neutrophils are loaded.

To do so, in some implementations, a syringe (for example, 1 mL syringe) can be filled with the chemokine solution 140 (for example, a solution of fMLP and fibronectin), and can be connected to the inlet 125 of the device. At this time, the outlet 130 can be blocked. By applying pressure to the syringe, the solution 140 can be flowed into the main channel 105. The pressure of the syringe can cause the chemokine solution 140 to displace the air in the side channels. In this manner, the air can be diffused out of the side channels through the PDMS. Once all the air has been displaced out of the side channels, both the main channel 105 and the side channels (110, 112, 114, 116, 118, 120) can be filled with the chemokine solution 140 (FIG. 1C).

In some implementations, after the chemokine solution 140 is flowed through the main channel 105, the inlet 125 and the outlet 130 can be closed. The chemokine solution 140 can wick into the side channels by capillary action. In some implementations, the microfluidic device 100 can be filled with a gas that dissolves in the chemokine solution 140 prior to flowing the chemokine solution 140 through the device 100. The gas can displace the air in both the main channel and the side channels. Subsequently, when the chemokine solution 140 is flowed through the main channel 105, the gas in the side channels will dissolve in the chemokine solution 140, and the chemokine solution 140 will flow into the side channels, for example, by capillary action.

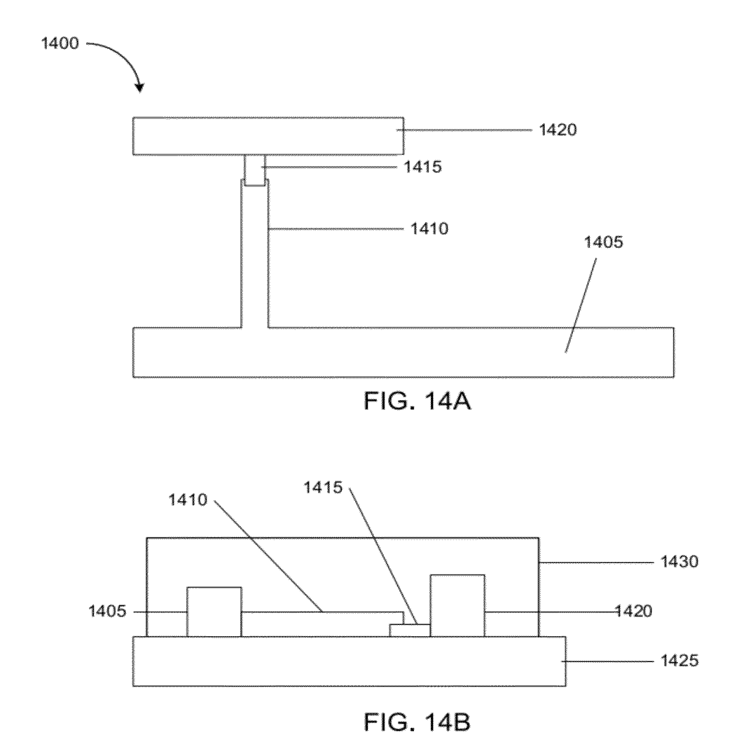

In some implementations, the chemokine solution 140 can be diffusively loaded directly into the side channels. In some implementations, reservoirs filled with chemokine solution 140 can be connected to the ends of the side channels through micro-valves. The micro-valves can be active or passive, and can permit the transfer of the chemokine solution 140 from the reservoirs directly into the side channels. An example of a passive micro-valve configured to allow transfer of chemokine solution 140 from the reservoirs to the side channels is described with reference to FIG. 14.

After filling the side channels and the main channel 105 with the chemokine solution 140, the main channel 105 can then be flushed with a wash buffer 145. In this manner, the chemokine solution from the main channel 105 can be removed from the microfluidic device 100 whereas the chemokine solution 140 in the side channels (110, 112, 114, 116, 118, 120) remains in the microfluidic device 100. The wash buffer can be the cell suspension itself.

Subsequently, a fluid suspension that is free of chemokines and includes the cells of interest (e.g., neutrophils) can be flowed into the main channel 105. The neutrophils can be obtained from blood samples drawn from healthy volunteer donors, for example, adults who are 18 years or older and who are not on any immuno-suppressants. For example, three 1 mL samples can be drawn from each volunteer with a time lapse of at least one week between draws.

In addition, neutrophils can be obtained from blood samples drawn from burn patients. For example, the blood samples can be drawn from burn patients who have sustained if burns cover at least 20% of their total body surface area. For example, a first 1 mL sample can be obtained within 72 hours after burn injury. Subsequently, two more 1 mL samples can be drawn at 48 hour intervals afterwards. Samples may not be drawn within 24 hours of an operative procedure.

Neutrophils can be isolated from blood samples using known techniques. For example, using sterile technique, neutrophils can be isolated from whole blood by density gradient separation using Polymorphprep (13.8% sodiuk diatrizoate and 8.0% polysaccharide, Axis-Shield, Rodelokka, Oslo, Norway), with centrifugation at 500 g for 40 minutes. To return the cells to an isotonic environment, the cells can be harvested and re-suspended in 10 mL of 0.5×PBS, then isolated by centrifugation at 400 g for 10 minutes. The neutrophils can be re-suspended in 50-100 μL of 1×PBS before loading into the microfluidic device 100. Samples can be processed within one hour after each blood draw and can be maintained at 37° C.

Once the neutrophils have been isolated from the blood and re-suspended in a fluid (for example, 1×PBS), the fluid suspension 150 that includes the neutrophils 155 can be infused into the device. To do so, the fluid suspension 150 can be introduced into the main channel 105, and the inlet 125 and the outlet 130 can be closed, for example, clamped. Notably, the fluid in which the neutrophils are suspended (for example, 1×PBS) is chemokine free. When the fluid suspension 150 is introduced into the main channel 105, the neutrophils 155 can settle, for example, at the bottom of the main channel 105 (FIG. 1E).

Because a side channel (for example, side channel 110) is filled with a chemokine solution and the main channel 105 is filled with a chemokine free solution, a chemokine concentration gradient is established within the microfluidic device 100. It is known that neutrophils experience a chemotactic effect in the presence of the chemokine concentration gradient, and consequently migrate from a region of lower chemokine concentration to a region of higher concentration. Consequently, the neutrophils 155 migrate through the side channels away from the main channel 105.

The microfluidic device 100 can be formed such that when several neutrophils 155 are introduced into the device, one neutrophil 155 migrates through each side channel. For example, the main channel 105 has a cross-section that is sufficient to allow the neutrophils 155 to be flowed through. Each side channel (110, 112, 114, 116, 118, 120) has a cross-section that is either as large as or smaller than a size of the neutrophil 155. Thus, when a neutrophil 155 enters, for example, side channel 110, the neutrophil 155 forms a plug within the side channel 110.

As a plug, the neutrophil 155 entraps the chemokine solution 145 in the region of the side channel 110 between the neutrophil 155 and the dead end of the side channel 110 (FIG. 1F). The chemotaxis effect causes the neutrophil 155 to continuously migrate in the direction of the entrapped chemokine solution 145. Further, because the concentration gradient on either side of the neutrophil 155 within the side channel remains substantially constant, neutrophil migration is improved.

By selecting the cross-section of the side channel 110 to be less than or equal to a size of the neutrophil 155, neutrophil migration in the absence of an active mechanism (for example, micro-pump, micro-valve, and the like) can be achieved. A photograph of an example of a microfluidic device 100 including a main channel 175 filled with neutrophils suspended in a chemokine-free solution is shown in FIG. 1G. As shown in the figure, multiple side channels (for example, 180, 182, 184) branch from the main channel. Each side channel has a corresponding inlet formed on the side wall of the main channel 175. Within each side channel, a neutrophil 190 migrates away from the main channel 175 due to the chemotaxis effect described previously. Notably, the neutrophils 190 migrate based on the chemokine concentration gradient and in the absence of active mechanisms. Consequently, the microfluidic device 100 is simple in construction and easy to use in prognostic and diagnostic applications.

FIG. 2 is a schematic of an example of a first implementation of a microfluidic device having a main channel 205 and multiple side channels (210, 212, 214, and 216). The main channel 205 can be of sufficient length to accommodate multiple side channels. For example, the main channel 205 can have a length of about 100 μm to 1 cm. The side channels (210, 212, 214, and 216) can each have a corresponding first inlet (220, 224, 228, and 232) formed in a side wall of the main channel 205, and can terminate in corresponding dead ends (222, 226, 230, and 234). In some implementations, each side channel can have a rectangular cross-section with 3 μm and 6 μm sides. Alternatively, or in addition, the side channel dimensions can range between 100 nm to 100 μm. Alternatively, the main channel or the side channel or both can have other cross-sections including round, circular, triangular, semi-circular, and the like. Each side channel can be 1000 μm long. Other dimensions are possible for the main channel 205 and each side channel. Further, the dimensions of the side channels can be the same as or different from each other. In some implementations, the size of the main channel can be significantly larger (about 10 times to 100 times) than the side channels so that the main channel can function as a sink.

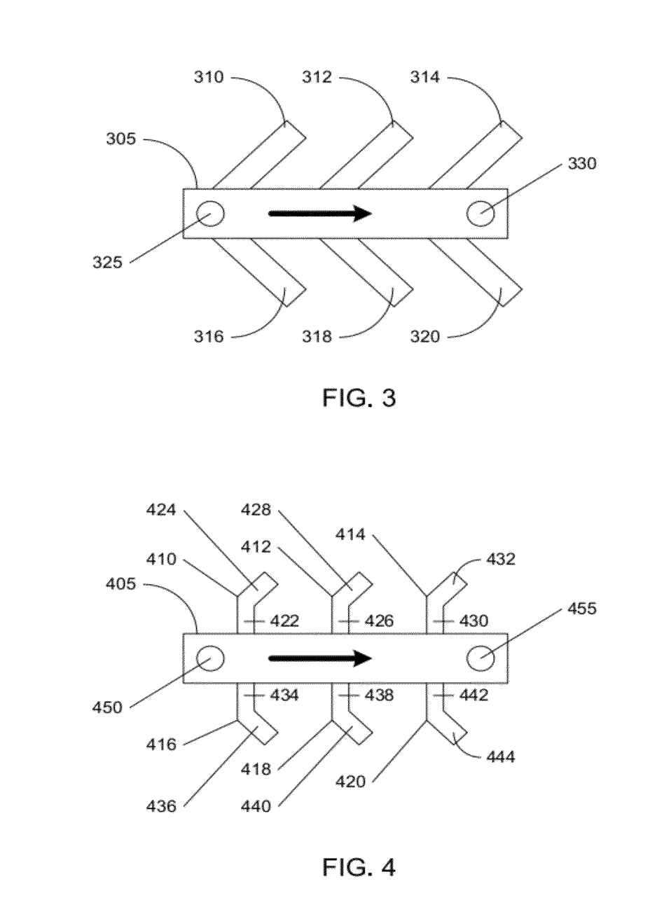

FIG. 3 is a schematic of an example of a second implementation of a microfluidic device having a main channel 305 and multiple side channels (310, 312, 314, 316, 318, and 320). The side channels of the microfluidic device described with reference to FIGS. 1A-1G were formed substantially perpendicular to the main channel. In alternative implementations, as shown in FIG. 3, the side channels (310, 312, 314, 316, 318, and 320) can be formed at an angle to the wall of the main channel 305. For example, each side channel can be inclined in a direction of flow of fluid from the inlet 325 to the outlet 330. Alternatively, some or all of the side channels can be inclined in a direction opposite to the flow of fluid. In some implementations, each side channel can be a straight channel. Alternatively, or in addition, each side channel can be a sigmoidal channel that has a wave shape.

FIG. 4 is a schematic of an example of a second implementation of a microfluidic device having a main channel 405 and multiple side channels (410, 412, 414, 416, 418, and 420). A side channel, for example, side channel 410, can include two portions—a first portion 422 and a second portion 424. The second portion 424 can be formed at an angle relative to the first portion 422. The first portion 422 can be connected to the main channel 405 and the second portion 422 can include the dead end. Similarly, all the side channels can include corresponding first portions (426, 430, 434, 438, and 442) and corresponding second portions (424, 428, 432, 436, 440, and 444). In some implementations, the first portions can be perpendicular to the main channel 405 and the second portions can be inclined relative to the first portion in a direction of flow of fluid from the inlet 450 to the outlet 455.

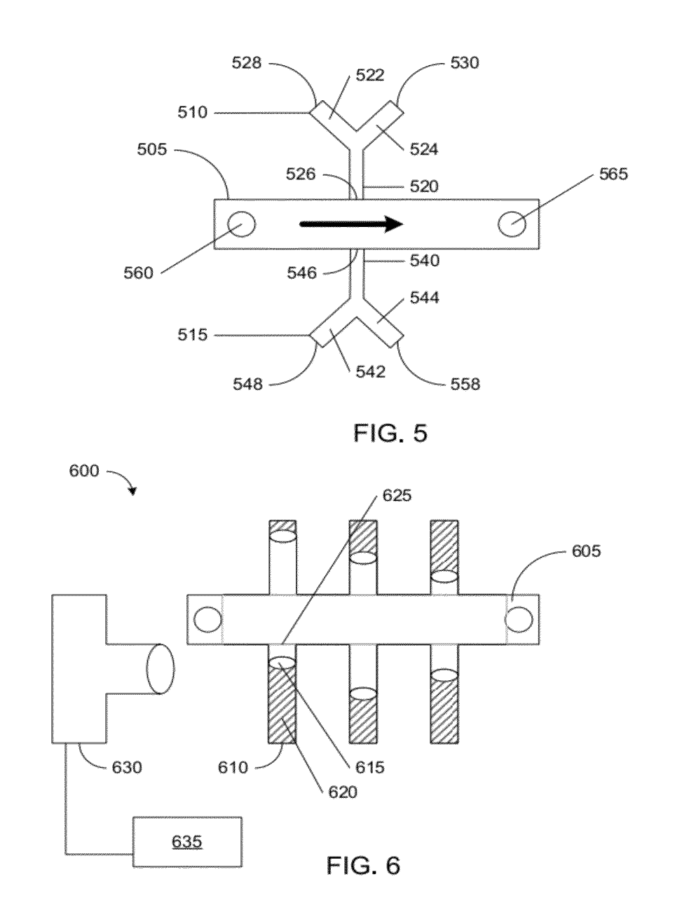

FIG. 5 is a schematic of an example of a third implementation of a microfluidic device having a main channel 505 and multiple side channels (510 and 515). Each side channel (for example, side channel 510) includes a first portion 522 and multiple second portions (for example, two second portions—522 and 524). The first portion includes an inlet 526 that is formed in a wall of the main channel 505. Both second portions (522 and 524) include a corresponding closed end (528 and 530, respectively). An end of the first portion is connected to an end of each second portion. Various shapes of the side channels can be beneficial when imaging cell motility. For example, the side channel configuration can allow imaging the entrance and end of a side channel and can also assist in cell loading.

FIG. 6 is a schematic of an example of a system 600 for determining migration speeds of neutrophils. The system 600 includes a microfluidic device that is similar to the microfluidic device 100 described with reference to FIGS. 1A-1F. The microfluidic device includes a main channel 605 and multiple side channels (for example, side channel 610). Neutrophils 615 migrate through the side channels, which have previously been filled with a chemokine solution 620, due to a chemotaxis effect created by a chemokine concentration gradient, as described previously.

The system 600 further includes an imaging system 630 configured to capture images and/or video of the neutrophil migration. For example, the imaging system 630 is configured to perform time-lapse imaging, and can include a Zeiss Axiovert microscope operating at a 20× magnification. One example of an imaging system is the Nikon Biostation IM, which is a system for time-lapse imaging with heated and humidified environmental chamber. The humidified environmental chamber increases the observation duration, for example, 24 hours for observing the migration of cancer cells towards serum factors.

The system 600 further includes a computer system 635 that is operatively coupled to the imaging system 630. The computer system 635 can include a computer-readable storage medium (for example, a hard disk, a CD-ROM, and the like) that stores computer program instructions executable by data processing apparatus (for example, a computer system, a processor, and the like) to perform operations. The operations can include controlling the imaging system 630 to capture images of the migration of neutrophils 615 through the side channels. In addition, the computer system 635 can receive the captured images from the imaging system 630, and process the images to obtain a migration speed of a neutrophil in a channel.

Alternatively, or in addition, neutrophils can be manually tracked to obtain migration rates. For example, migration rates of neutrophils (for example, 50 neutrophils/sample) can be tracked, and the migration rates can be calculated using Image J (NIH) software executed by the computer system 635. In some situations, experiments to characterize the formation of gradients inside the device in the absence of neutrophils can be performed by replacing all or portions of the chemokine solution (for example, the fMLP) with fluorescein (Sigma) of comparable molecular weight, and analyzing the distribution and changes in fluorescence intensity from time-lapse imaging using the imaging system 630 and the computer system 635. Further, the computer system 635 is configured to execute computer software applications that perform statistical analysis of the data captured by the imaging system 630. For example, the computer system 635 is configured to execute the Shapiro-Wilk test to test the normality of the distribution of migration speed values for neutrophils in the same sample. The test indicates if the data is likely to be derived from a normally distributed population (p>0.05). The computer system 635 is further configured to perform multivariate analysis to determine correlations between neutrophil migration speed and clinical parameters.

The microfluidic devices described herein can be used to analyze different types of motile cells including neutrophils. Some of the different types of cells, components of a chemokine solution in which each cell experiences a chemotaxis effect, cell sizes, and corresponding side channel sizes, are shown in Table 1.

TABLE 1

| | | Cell Diameter | Channel Size |
|---|---|---|---|
| Cell type | Chemoattractants | [μm] | [μm] |
| Neutrophils | IL8, fMLP, LTB4, | 9-12 | 3 × 6 |
| | C5a | | |
| T Lymphocytes | SDF1, CXCL10, | 7 | 6 × 6 |
| | CCL19, | | |
| Dendritic Cells | CCL19, CCL21 | 10-15 | 8 × 8 |
| Monocytes | MCP1, CCL7 | 10-30 | 8 × 8 |
| Eosinophils | eotaxin, RANTES, | 9-14 | 6 × 6 |
| | MCP-3, MCP-4, | | |
| | CCR3 | | |
| NK cells | MIP-1 alpha, MCP- | 8-10 | 6 × 6 |
| | 1, RANTES, | | |
| | CXCL14 | | |
| B cells | SDF1, CCL11 | 8 | 6 × 6 |
| Lymphoblast | CCL22, MCP1 | 12-20 | 8 × 8 |
| Reticulocytes | SDF1 | 7-12 | 3 × 6 |
| Platelets | Collagen | 1-4 | 0.5 × 1 |
| Circulating Tumor | SDF1 | 7-40 | 10 × 10 |
| Cells (CTCs) | | | |
| Circulating | VEGF, FGF, NO, | 10-20 | 8 × 8 |
| Endothelial Cells | S1P | | |
| (CECs) | | | |
| Bacteria | | 0.1-1 | 0.1-1 × 2 |

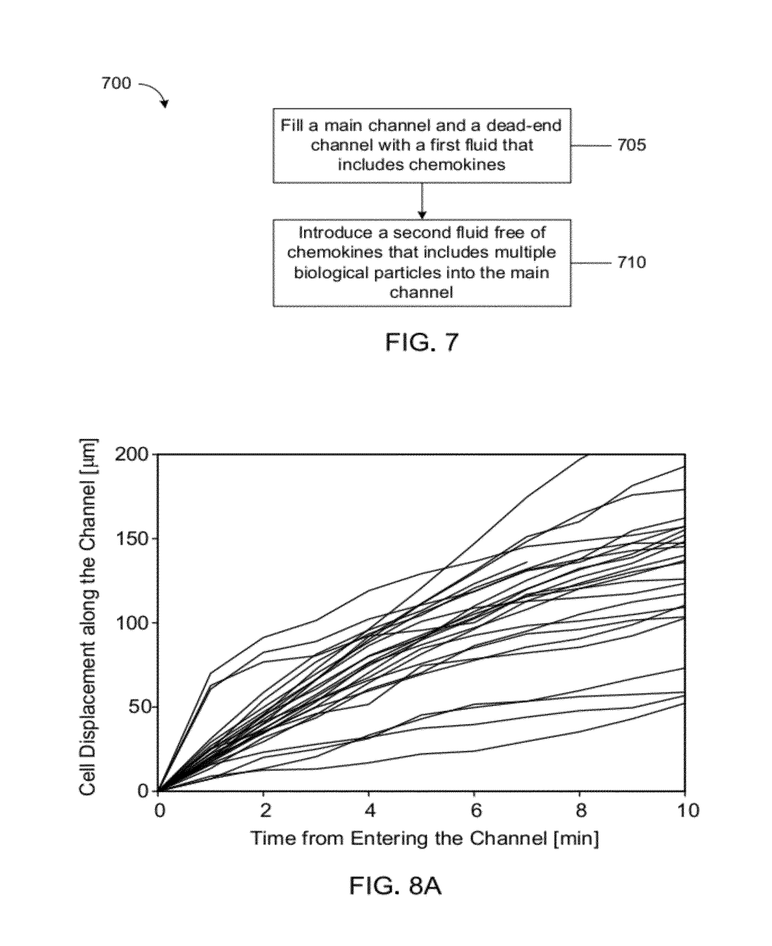

FIG. 7 is a flowchart of an example of a process 700 for detecting neutrophil motility using a microfluidic device. In a microfluidic device similar to the device described with reference to FIGS. 1A-1F, a main channel and a side channel are filled with a fluid that includes chemokines (705). A second fluid that is free of chemokines and that includes cell (for example, neutrophils) is introduced into the main channel (710). The chemotaxis effect causes migration of a neutrophil through the side channel at a motility (i.e., a migration rate) that can be measured.

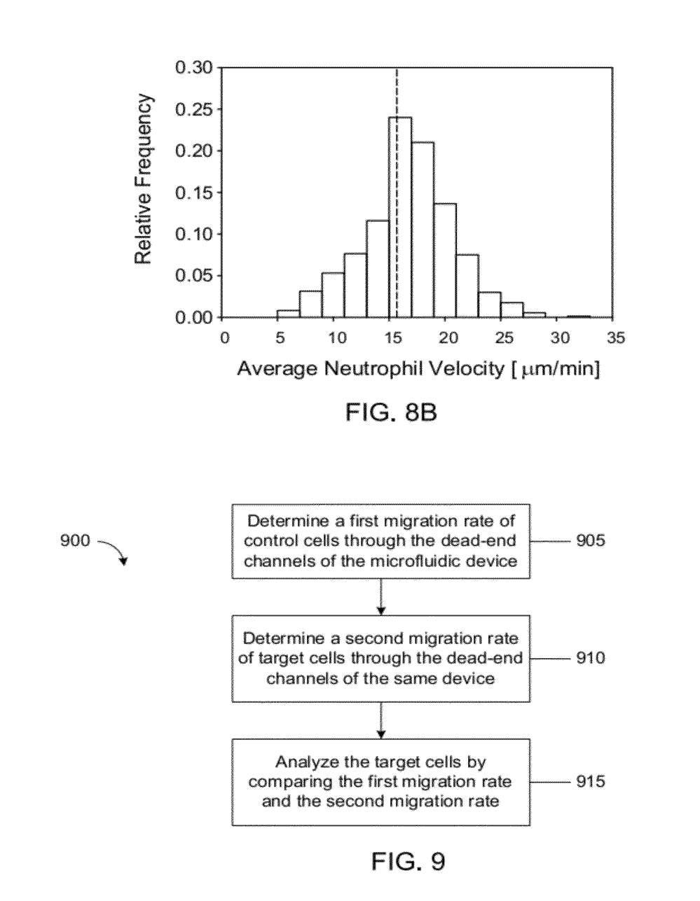

FIGS. 8A and 8B are results of device validation experiments. FIG. 8A is a plot of displacement of multiple neutrophils along corresponding channels within the microfluidic device over time. Most of the neutrophils maintain constant speed in the direction of higher chemokine concentration within the channels for at least the first 10 minutes. A few neutrophils moved faster for the first 100 μm inside the channels, decelerated later, and maintained constant speed thereafter. Moving neutrophils displayed persistent behavior along the channels, with no changes in the direction of migration.

FIG. 8B is a bar graph that shows the distribution of average speed of migration for 800 neutrophils from one healthy donor. The average speeds of all cells were calculated and the normality of distribution around the mean of the velocities of the almost 800 cells moving through the side channels was verified. When compared with the average migration speed of 50, 100 and 200 cells randomly chosen at different locations throughout the device, no statistically significant difference between the four populations was observed. In additional control experiments, in the absence of fMLP chemokine, neutrophils did not migrate through the side channels of the device. Furthermore, when a sample of neutrophils was diluted ten-fold, the measured average migration speed was unchanged (17±6 μm/min and 18±4 μm/min, p=0.3), suggesting that migration speed was independent of the concentration of neutrophils in the device.

FIG. 9 is a flowchart of an example of a process 900 for analyzing migration rates of motile cells. The process 900 can be implemented to analyze target cells (for example, neutrophils obtained from burn patients) by comparing the target cells with control cells (for example, neutrophils obtained from healthy donors). By implementing the process 700 described with reference to FIG. 7, a first migration rate of control cells through the side channels of the microfluidic device can be determined (905). Subsequently, a second migration rate of target cells through the side channels of the same microfluidic device can be determined (910). The target cells can be analyzed by comparing the first migration rates of the controls cells and the second migration rates of the target cells (915).

The devices can be single-use devices. In other situations, the devices can be re-used by lysing the neutrophils, for example, with distilled water, or some detergents, and removing the cell debris by persistent washing. In particular, if the side channels are connected to reservoirs through micro-valves, then the side channels can be washed by pumping suitable fluids into the side channels through the valves. Also, migration of control and target cells can be performed in the same device. Alternatively, control cell migration can be measured using a first device and target cell migration can be measured using a second, separate device.

In one implementation of process 900, a total of 23 blood samples were collected from 18 healthy volunteers aged 19-68 years (mean 40 years) (Table 2); 61% of volunteers were female, and 39% male. Volunteers had no past medical history with the exception of hypertension, hypothyroidism, and depression. The most common medications taken were oral contraceptives and antidepressants. No volunteer took immunosuppressant drugs.

TABLE 2
Clinical overview of burn patients' data

| | Volunteers | Patients |
|---|---|---|
| No. Participants | 18 | 8 |
| Average Age in Years | 40 | 31 |
| (Range) | (19-68) | (1-56) |
| Female | 11 | 2 |
| (%) | (61) | (25) |
| Male | 7 | 6 |
| (%) | (39) | (75) |
| Caucasian | 17 | 5 |
| (%) | (94) | (63) |
| Hispanic | 0 | 3 |
| (%) | | (37) |
| Asian | 1 | 0 |
| (%) | (6) | |
| No. without | 10 | 5 |
| Comorbidities (%) | (56) | (63) |
| Comorbidities | Depression | Depression |
| (in descending order of | Hyperlipidemia | Hyperlipidemia |
| frequency) | Hypertension | Hypertension |
| | Hypothyroidism | Cirrhosis |
| Medications | Oral contraceptives | ACE Inhibitors |
| (in descending order of | SSRIs | Benzodiazepines |
| frequency) | ACE Inhibitors | |
| | Diuretics | |
| | Thyroid replacement | |
| | therapy | |
| | Statins | |
| | Proton pump inhibitors | |
| No. with Inhalation | N/A | 3 |
| Injury | | (13) |
| (%) | | |
| No. Requiring | N/A | 13 |
| Mechanical Ventilation | | (54) |
| (%) | | |
| No. with Positive Blood | N/A | 1 |
| Cultures <72 hrs Post- | | (13) |
| Burn (%) | | -Acinetobacter- |
| -Microbes- | | |
| No. with Positive Blood | N/A | 4 |
| Cultures 1-4 Weeks | | (50) |
| Post-Burn (%) | | -Enterococcus, |
| -Microbes- | | MRSA, MSSA, |
| | | Enterobacter, |
| | | Candida- |
| No. with Positive | N/A | 2 |
| Sputum Cultures <72 hrs | | (25) |
| Post-Burn (%) | | -MSSA, H. influenzae- |
| -Microbes- | | |
| No. with Positive | N/A | 2 |
| Sputum Cultures 1-4 | | (25) |
| Weeks Post-Burn (%) | | -E. coli, MRSA- |
| -Microbes- | | |

Average neutrophil migration speed for all volunteer samples was 18±5 μm/min (range 14 to 24 μm/min) (FIG. 8B).

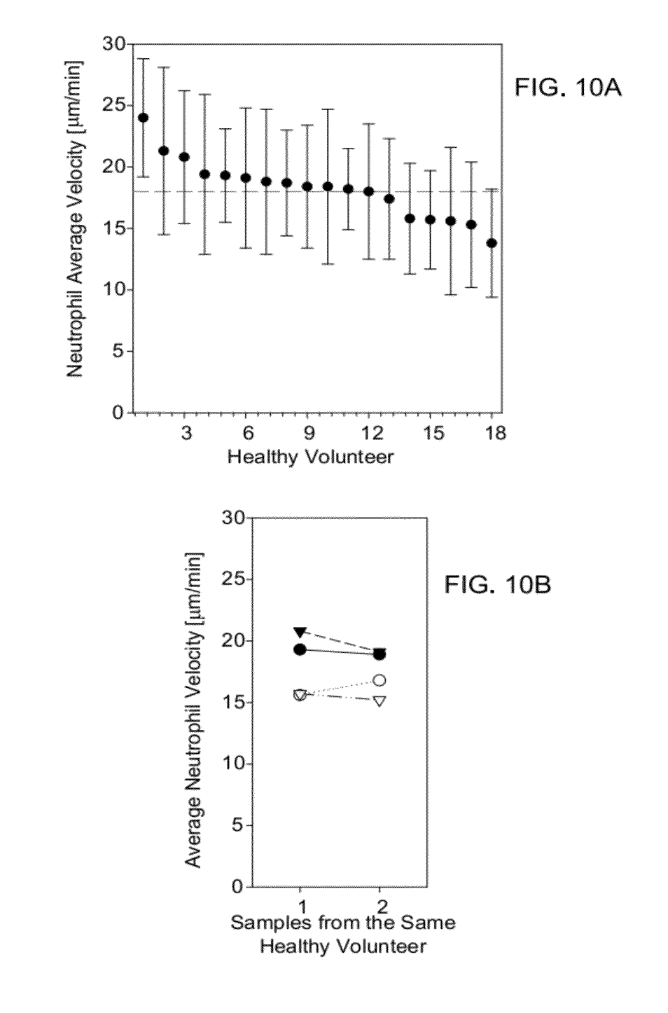

FIGS. 10A and 10B are a plot of average neutrophil motility in 18 healthy donors and a validation of the repeatability of neutrophil motility, respectively. To verify the reproducibility of the neutrophil migration speed measurements, the experiments were repeated with neutrophils isolated from four healthy volunteers, with samples drawn between one week and one month apart. The experiment yielded equivalent neutrophil velocities, thus suggesting high reproducibility (FIG. 10A, p≧0.05 in all cases). There were no correlations between the neutrophil migration speed and the volunteer age or gender (FIG. 10B).

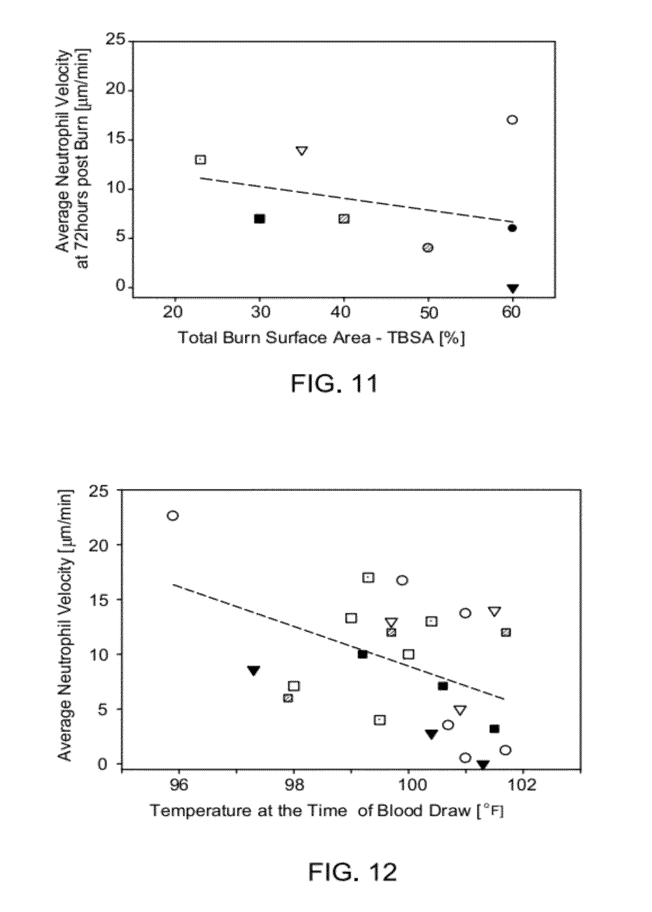

FIG. 11 is a plot showing a correlation between neutrophil motility and total burn surface area in burn patients. Neutrophil motility was measured in a total of 24 samples from 2 pediatric and 6 adult patients (mean age 23 years, range 1-48 years) after sustaining 20-60% TBSA burns (Table 2); 63% of patients were male, and 38% were female. The most common co-morbidities were depression and hypertension. No patient took immunosuppressant drugs. As shown in FIG. 11, the average neutrophil migration speed in burn patients was 9±6 μm/min, significantly lower than in controls (p<0.01). In patients who were not bacteremic at 72-120 hours post-injury, neutrophil migration speed was negatively correlated with severity of burn injury (FIG. 11, R²=0.6).

FIG. 12 is a plot showing a correlation between neutrophil motility and blood temperature. As shown in FIG. 12, across the entire data set for burned patients, there was poor correlation between neutrophil migration speed and temperature (R²=0.2). Poor correlation was also observed between neutrophil motility and absolute neutrophil count (R²=0.23), and neutrophil motility and the percentage of band cells in the neutrophil population (R²=0.01).

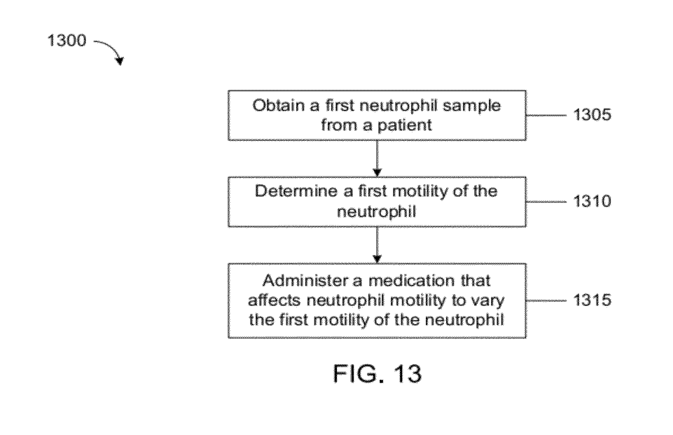

FIG. 13 is a flowchart of an example of a process 1300 for altering neutrophil motilities in a patient. A neutrophil sample is obtained from a patient (1305). For example, the neutrophil sample is obtained from the blood sample of a burn patient. A motility of the neutrophil is determined (1310). For example, using the microfluidic device, detection system, and computer system described previously, a neutrophil motility (i.e., a neutrophil migration rate) is obtained. A medication that affects neutrophil motility can be administered to the patient to vary the measured motility (1315). The medications can include one or more of several modulators of neutrophil migration. Such modulators—some produced inside the body, some from external sources or chemicals/drugs—include endogenous modulators (for example, acetylcholine, IL10, TNFalpha, IL1, IL6, and the like) and exogenous modulators (for example, curcumin, lysophosphatidylglycerol, cigarette smoking, cholinergic drugs).

Thus, the microfluidic device can be applied to the analysis of neutrophil motilities in burn patients. In some situations, neutrophils obtained from burn patients can be studied over a one week period, for example, at 48 hour intervals. To determine a long-term effect of the burn injury and treatment on the patients, the study period can be expanded to six months. In such situations, the neutrophil motility of the patients can be determined periodically. For example, for burn patients, neutrophil motility can be measured daily for the first two weeks and then weekly for the subsequent six months. For transplant patients, motility can be monitored on a monthly basis.

Different types of devices can be used at the different monitoring stages, thereby yielding different information. For example, monitoring the ability of T-helper lymphocytes during immuno-suppressive therapy would have the goal of identifying the minimal drug doses that keep the lymphocytes from moving too fast, and potentially preventing rejection. Monitoring the neutrophils would have the goal of identifying the maximal drug dose to identify the risk of too much immune suppression, which can increase the risk of infections.

Recovery for burn patients takes significantly longer than one week after the burn injury. Further, burn patients are susceptible to infections for life. Periodic studies of neutrophil motilities over a six month period can enable determining an effect of the treatment. In particular, neutrophil motility can be monitored to determine if there is a correlation between motility and infections. For example, if the neutrophil motility of the patient decreases over time, then it can be determined that the patient is at a higher risk of infection, and a treatment can be administered accordingly.

Similarly, neutrophil motility can be studied to determine if a correlation exists between motility speed and rate of wound healing. For example, if neutrophil motility increases over time and the patient's wounds are healing, then a rate of migration speed that indicates wound healing can be determined. In another burn patient, if the rate of migration speed does not match the rate previously determined as indicating wound healing, then a treatment can be administered to alter, i.e., increase the neutrophil migration speed.

Although neutrophil mobility is not the sole indicator of infections or healing in burn patients, it is a significant indicator. Because the microfluidic device described above enables easy determination of neutrophil mobility, a range of migration speeds that indicate wound healing or heightened risk of infection or both can be determined. Based on these determinations, treatments unique to patients can be tailored.

Further characterization of neutrophil chemotaxis using this microfluidic device may have important diagnostic implications not only for burn patients but also for patients afflicted by other diseases that compromise neutrophil functions. For example, the device can be applied to analyze neutrophil motility in pediatric patients to identify patients who are at a higher risk for certain diseases. In transplantations, the device can be used to analyze neutrophil motilities to determine if there is a correlation between neutrophil motility under medication and the occurrence of complications, for example, infections and rejections. By determining a range of neutrophil motilities that correlate to low infection and at which immuno-suppressant functions are not suppressed, it may be possible to vary the quantities of immuno suppressant medication that is being administered to patients.

Neutrophil motility measured using this device can be one metric for identifying a treatment in patients suffering from fungal infections. Because such patients are critically ill, doctors often rely upon intuition to immediately start a course of treatment while waiting on test results that can take between 36 and 72 hours to obtain. Because neutrophil motility is believed to decrease due to fungal infections but not due to bacterial infections, motilities determined using this device can be used to determine that the infections are fungal, and then, as one metric in a selection of a course of treatment.

FIGS. 14A-B are schematics of an example of a passive micro-valve 1400. As shown in a top view (FIG. 14A), the micro-valve 1400 connects a side channel 1410 to a chemokine solution channel 1420 through a metal patch 1415. The micro-valve 1400 can be operated to allow chemokine solution flowed through the channel 1420 into the side channel 1410. As shown in a side view (FIG. 14B), the main channel 1405, the side channel 1410, the metal patch 1415, and the chemokine solution channel 1420 can be formed on a substrate 1425, for example, a glass substrate. A layer 1430, for example, of PDMS, can be formed on the substrate 1425 to cover substantially all the channels and the metal patch 1415.

The metal patch 1415 prevents the layer 1430 from adhering to the substrate 1425. When the pressure in the chemokine solution channel 1420 is increased above a certain threshold, leakage will occur between the layer 1430 and the metal patch 1415. This will create an opening between the channel 1420 and the side channel 1410. Relieving the pressure will close the opening, stop the flow, and restore the sealing. In some implementations, the layer 1430 can be made from PDMS or any elastic material that is flexible under high pressure and retains its original shape upon relief of the pressure. The pressure required to open and close the micro-valve 1400 can depend on the side of the metal patch 1415 (for example, width and length) and the elasticity of the layer 1430. In addition, the pressure can also be affected by the stickiness of the layer 1430.

While this specification contains many specific implementation details, these should not be construed as limitations on the scope of any disclosures or of what may be claimed, but rather as descriptions of features specific to particular implementations of particular disclosures. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Thus, particular implementations of the subject matter have been described. Other implementations are within the scope of the following claims.

The specification discloses methods, apparatus, and systems for analyzing neutrophil motility using microfluidic devices. Specifically, in some implementations, motility of a neutrophil in a chemokine concentration gradient is described. It will be appreciated that the microfluidic device and the techniques described herein can be used to detect motility of any type of cell in an environment in which the cell will migrate due to a concentration gradient. For example, the concentration gradient can be created using a light source such that the cell migrates from a region of low light intensity toward a region of high light intensity (or vice versa). In another example, the concentration gradient can be created using a magnetic source such that the cell migrates from a region of low magnetic field toward a region of high magnetic field. Thus, in general, the motility of cells that migrate in the presence of any type of concentration gradient can be studied by flowing a fluid including such cells into the microfluidic device having the main channel and the branching side channels. The universe of cells and organisms that can be tested in such a microfluidic device can include cells and organisms that have one or more cilia (for example, bacteria, sperm, and the like) that have sizes sufficient to be mechanically restricted within one of the branching side channels.

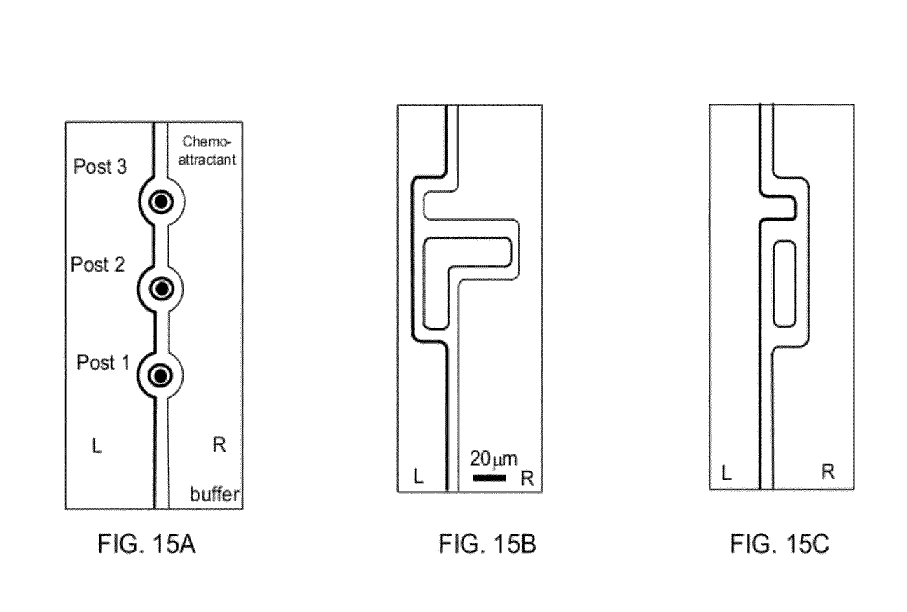

FIGS. 15A-C are schematics of examples of bifurcated side channels. Bifurcating channels and post geometries can quantify the directional decision making during the migration of neutrophils. For example, neutrophils from a healthy donor will pick the shorter path (e.g., marked “L” in FIG. 15B) when migrating through an asymmetric path. Neutrophils from burn patients will show different defects, and may stop either at the bifurcation and not be capable of moving forward or pick the longer path (e.g., marked “R” in FIG. 15B) with higher frequency, or even go around (through the path marked “L”, and then the path marked “R”) or circle between the two paths. Preliminary results show that at two weeks after burn injury, neutrophil migration speed towards fMLP recovers to normal reference values. However, the orientation of the neutrophils in bifurcating channels is still severely impaired. The device allows the characterization of neutrophil orientation at single cell resolution and with high temporal resolution. This allows one to distinguish between subpopulations of normal and defective neutrophils in the circulation and quantify the relative size of each.

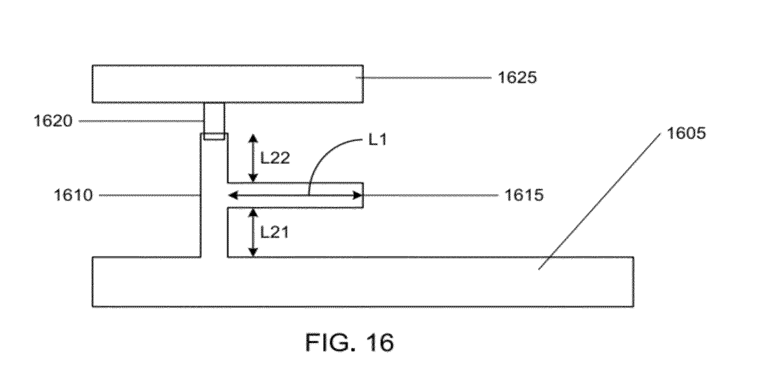

FIG. 16 is a schematic of an example of a system for switching chemokine gradients. The system includes a main channel 1605 and a chemokine solution channel 1625 connected by a passive micro-valve 1620 and a side channel 1610 that includes a bifurcating channel 1615. The side channel 1610 and the bifurcating channel 1615 can be filled with two distinct solutions (for example, a chemo-attractant solution and an inhibitor solution), which will reach the neutrophils migrating through the two channels at different times during the migration.

In some implementations, all the channels (including the side channel 1610 and the bifurcating channel 1615) can be filled with a first chemokine solution. A second chemokine solution can be introduced through the chemokine solution channel 1625 into the side channel 1610 through the micro-valve 1620. When the pressure in the chemokine solution channel 1625 is increased, the second chemokine solution flows into the side channel 1610 without washing the first chemokine solution through the bifurcating channel 1615. Cells in suspension can be introduced in the main channel, removing any excess of the second chemokine solution.

The cells migrate through the side channel 1610 in a first chemokine solution environment. When the cells reach the entrance of the bifurcating channel 1615, then the cells diffuse from the side channel 1610 into the bifurcating channel 1615. The geometry and arrangement of the side channel 1610 and the bifurcating channel 1615 can be selected to regulate the timing and temporal changes of the two chemokine solutions. In some scenarios, the first chemokine solution can be a drug. An array of bifurcating channels on both sides of each side channel can be used. By changing the ratios between the dimensions shown in FIG. 16, and by changing the width and shape of the side channel 1610 and the bifurcating channels 1615, the temporal patterns of sequential stimulation can be adjusted.

In some implementations, neutrophil chemotaxis analysis can be performed using whole-blood without separating the neutrophils from the blood. Such a system can offer several potential advantages such as, for example, less than five minutes from blood draw to moving neutrophils, minimal risks for activating/altering neutrophils during sample separation (due to absence of density-based separation media, anti-body capture, E-selecting capture, electrical or other physical fields), low blood volume (for example, 10 μL or less.

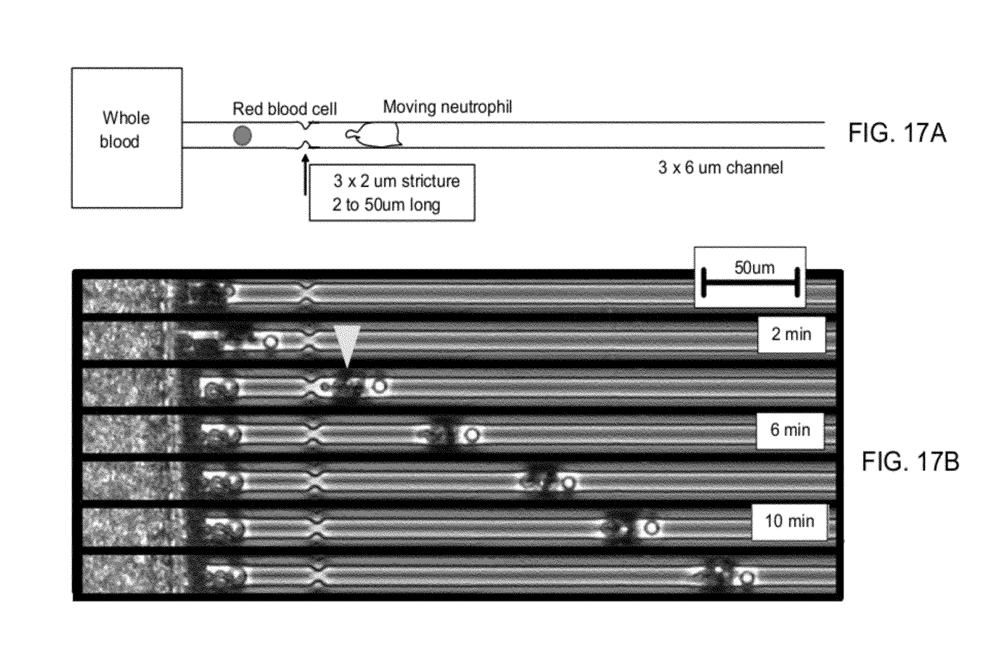

FIG. 17A is a schematic of an example of a channel for analyzing chemotaxis migration from whole blood. FIG. 17B is a photograph of an example channel used for analyzing chemotaxis migration from while blood. The feature that enables the use of whole blood in the device is represented by the small constriction at the entrance to the side channels, as shown in FIG. 17A. The size and shape of this stricture is such that during whole blood loading in the main channel, red blood cells cannot pass into the side channels. The pressure required for pushing the blood into the main channel during loading is less than the pressure that would deform the red blood cells through the stricture. For example, the stricture can be 2 μm wide, 3 μm tall, and 4 μm long. Such a stricture is effective in preventing red blood cells (donut shaped, 6 μm diameter and 1 μm tall) from passing into the side channels. Neutrophils can squeeze through the stricture apparently with no consequences for their migration. In one sample of capillary blood from a healthy volunteer, the average migration speed was 17 μm/min, comparable with the average from healthy donors measured in the regular device. Neutrophils from human and animal blood could be analyzed.

Alternatively, strictures can have a length between 4 and 50 μm and can be effective in blocking the entrance of red blood cells but allowing the passage of neutrophils. The strictures can be smaller or larger than those described here, and can be used to analyze the migration of other cells in blood, for example, lymphocytes, monocytes, natural killer lymphocytes, platelets and megacariocytes, epithelial cells, endothelilal cells, cancer cells, and the like.

Occasionally, few red blood cells pass through the stricture, either during whole blood loading or pushed forward by the moving neutrophils. These red blood cells appear not to interfere with the neutrophil chemotaxis. When many red blood cells clog the entrance of a channel, neutrophils can have a difficult time entering those channels.

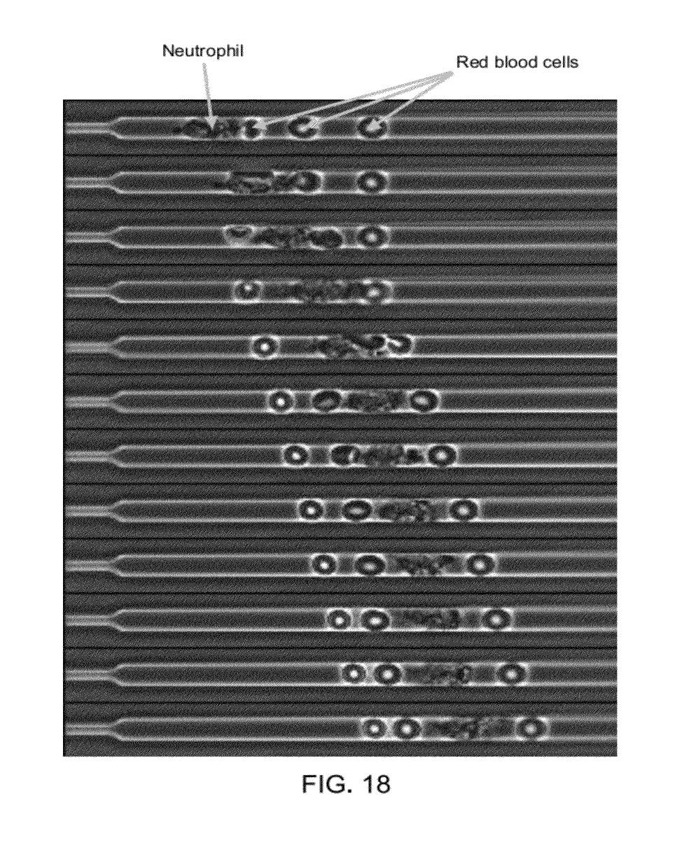

FIG. 18 is a photograph that shows neutrophils migrating from left to right in response to a chemo-attractant, namely, fMLP. Three red blood cells, initially in front of the neutrophil, were by-passed by the moving neutrophil. The red blood cells in front and behind the neutrophil appeared to be moving together with the neutrophil. One possible explanation could be that the moving neutrophils acted as a “piston” moving the fluid in front and behind it.

What is claimed is:

1. A method for manipulating cells in a chemo-attractant environment, the method comprising:
filling a main channel and a side channel branching off of the main channel with a first fluid that includes at least one chemokine; and
introducing a second fluid free of the at least one chemokine and that includes a plurality of cells into the main channel such that an individual cell of the plurality of cells can enter and form a plug in the side channel such that a chemokine concentration gradient is formed in the side channel on opposite sides of the individual cell.

2. The method of claim 1, wherein filling comprises applying pressure at the main channel inlet that is sufficient to move the first fluid through the main channel and into the side channel.

3. The method of claim 2, wherein applying pressure comprises applying sufficient pressure to displace air in the side channel through a gas permeable substrate defining a portion of the side channel.

4. The method of claim 1, further comprising filling the side channel with a gas that is soluble in the first fluid prior to filling the side channel with the first fluid.

5. The method of claim 1, wherein filling the side channel with the first fluid comprises flowing the first fluid through the main channel past the side channel inlet, wherein the first fluid fills the side channel by capillary action.

6. A device for analyzing migration of cells in a chemo-attractant concentration gradient, the device comprising:
a main channel having a pair of side walls, a main channel inlet, and a main channel outlet, wherein the main channel is sized to receive the cells; and
a side channel that branches from the main channel, the side channel having an inlet formed in a side wall of the main channel and terminating in a closed end, wherein the side channel is sized such that the cells can migrate from the main channel into and form a plug in the side channel and then move along the side channel in the presence of a chemo-attractant concentration gradient established in the side channel.

7. The device of claim 6, wherein the main channel and the side channel are positioned on a substrate comprising a gas-permeable material.

8. The device of claim 6, wherein the main channel has a cross-sectional area sufficient to allow the cells to flow through.

9. The device of claim 6, wherein the side channel has a cross-sectional area that is smaller than a cross-sectional area of the main channel.

10. The device of claim 6, wherein the side channel is rectangular in cross-section, and wherein sides of the rectangular cross-section are about 3 μm and 6 μm.

11. The device of claim 6, wherein the device further comprises a plurality of side channels each of which branches from the main channel and has a corresponding inlet formed on a side wall of the main channel.

12. The device of claim 11, wherein one or more of the plurality of side channels branch in a direction that is perpendicular to an axis of the main channel between the main inlet and the main outlet.

13. The device of claim 6, wherein the side channel is a straight channel.

14. The device of claim 6, wherein the side channel includes a bend between the first end and the second end.

15. The device of claim 6, wherein the side channel comprises a first portion that includes the inlet and a first end, and a plurality of second portions, each including a second end and terminating at a corresponding closed end, wherein each second end is connected to the first end.

16. A system for analyzing cells, the system comprising:
a device including:
a main channel having a pair of side walls, a main channel inlet, and a main channel outlet, and
a plurality of side channels, each side channel having an inlet connected to a side wall of the main channel, wherein each side channel terminates in a corresponding closed end, wherein cells are introduced through the main channel inlet and migrate through the plurality of side channels; and
a detection unit configured to detect a migration of the cells through the device.

17. The system of claim 16, wherein the device comprises a gas permeable substrate defining at least part of the side channels.

18. The system of claim 16, wherein a cross-sectional area of each side channel is less than a cross-sectional area of cells that migrate through the side channel.

19. The system of claim 16, wherein the main channel has a rectangular cross-section.

20. The system of claim 16, wherein a side channel is sized such that the cell forms a plug in the side channel.

21. A method for analyzing target cells, the method comprising:
determining a first migration rate of control cells through a side channel having an inlet and terminating in a dead end, wherein the control cells migrate through the side channel due to a chemokine concentration gradient, wherein the first migration rate includes a distance traveled by a control cell in the side channel per unit time;
determining a second migration rate of target cells through the side channel, wherein the target cells migrate through the side cells due to the chemokine concentration gradient, wherein the second migration rate includes a distance traveled by a target cell in the side channel per unit time; and
analyzing the target cells by comparing the first migration rate and the second migration rate.

22. The method of claim 21, wherein determining the first migration rate comprises:
filling the side channel with a first fluid that includes chemokines;
introducing a second chemokine-free fluid that includes the control cell at the inlet establishing a chemokine gradient in the side channel, wherein the control cell migrates towards a region of high chemokine concentration in the side channel; and
measuring the distance traveled by the control cell in the side channel per unit time.
